(19) United States
(12) Patent Application Publication
(10) Pub. No.: US 2021/0290610 A1
(43) Pub. Date: Sep. 23, 2021

(54) COMBINATION OF FXR AGONISTS

(71) Applicants: Andreas BAUER, Heidelberg (DE); Bryan LAFFITTE, Cardiff, CA (US); Patrick MUELLER, Bottmingen (CH)

(72) Inventors: Andreas BAUER, Heidelberg (DE); Bryan LAFFITTE, Cardiff, CA (US); Patrick MUELLER, Bottmingen (CH)

(21) Appl. No.: 16/332,446

(22) PCT Filed: Sep. 12, 2017
(86) PCT No.: PCT/IB2017/055503

§ 371 Date: Mar. 12, 2019

Related U.S. Application Data
(60) Provisional application No. 62/394,446, filed on Sep. 14, 2016.

(51) Int. Cl. A61K 31/46; A61K 31/4178; A61P 1/16

(52) U.S. Cl. CPC A61K 31/46; A61P 1/16; A61K 31/4178

(57) ABSTRACT

The invention provides pharmaceutical compositions comprising a farnesoid X receptor (FXR) agonist and another therapeutic agent, in particular for treating or preventing liver diseases or disorders.

### FIELD OF THE INVENTION

The present invention relates to a pharmaceutical combination comprising at least one farnesoid X receptors (FXRs) agonist and another therapeutic agent, in particular CCR2/CCR5 antagonist such as Cenicriviroc, optionally in the presence of a pharmaceutically acceptable carrier and pharmaceutical compositions comprising them. Furthermore, the invention is directed to the use of such pharmaceutical combinations for treating or preventing fibrotic or cirrhotic diseases or disorders, e.g. liver diseases or disorders, as well as compositions, methods, uses and regimens involving such combinations.

### BACKGROUND OF THE INVENTION

Farnesoid X Receptor Agonist (FXR) is a nuclear receptor activated by bile acids, also known as Bile acid Receptor (BAR). FXR is expressed in principal sites of bile acid metabolism, such as liver, intestine and kidney, where it mediates effects on multiple metabolic pathways in a tissue-specific manner.

The mode of action of FXR in the liver and intestine is well known, and described e.g. in (Calkin and Tontonoz, (2012), Nature Reviews Molecular Cell Biology 13, 213-24). FXR is responsible for modulating bile acid production, conjugation and elimination through multiple mechanisms in the liver and intestine. In normal physiology, FXR detects increased levels of bile acids and responds by decreasing bile acid synthesis and bile acid uptake while increasing bile acid modification and secretion in the liver. In the intestine, FXR detects increased bile acid levels and decreases bile acid absorption and increases secretion of FGF15/19. The net result is a decrease in the overall levels of bile acids. In the liver, FXR agonism increases expression of genes involved in canalicular and basolateral bile acid efflux and bile acid detoxifying enzymes while inhibiting basolateral bile acid uptake by hepatocytes and inhibiting bile acid synthesis.

Furthermore, FXR agonists decrease hepatic triglyceride synthesis leading to reduced steatosis, inhibit hepatic stellate cell activation reducing liver fibrosis and stimulate FGF15/FGF19 expression (a key regulator of bile acid metabolism) leading to improved hepatic insulin sensitivity. Thus, FXR acts as a sensor of elevated bile acids and initiates homeostatic responses to control bile acid levels, a feedback mechanism that is believed to be impaired in cholestasis. FXR agonism has shown clinical benefits in subjects with cholestatic disorders (Nevens et al., J. Hepatol. 60 (1 SUPPL. 1): 347A-348A (2014)), bile acid malabsorption diarrhea (Walters et al., Aliment Pharmacol. Ther. 41(1):54-64 (2014)) and non-alcoholic steatohepatitis (NASH; Neuschwander-Tetri et al 2015).

Bile acids are normally produced by the organism. At high dose they can cause different side effects as they have detergent properties (diarrhea or cellular injury). In addition, they can also cause pruritus.

C-C chemokine receptor type 2 (CCR2) and CCR5 play a role in entry of viruses such as Human Immunodeficiency Virus (HIV) into the cell, but also are important for the recruitment of immune cells to sites of injury. Inhibition of this receptors activity may have an anti-inflammatory effect. And recent data indicate that these receptors may also play a role in promoting hepatic fibrosis.

Cenicriviroc (also known as CVC) is (S,E)-8-(4-(2-butoxyethoxy)phenyI)-1-(2-methylpropyl)-N-(4-(((1-propyl-1H-imidazol-5-yl)methyl)sulfinyl)phenyl)-1, 2,3,4-tetrahydrobenzo[b]azocine-5-carboxamide. Cenicriviroc binds to and inhibits the activity of the C-C chemokine receptor type 2 (CCR2) and C—C chemokine receptor type 5 (CCR5) receptors.

Nonalcoholic fatty liver disease (NAFLD) is the most common cause of chronic liver disease in the Western world (Ratziu et al 2010). The main stages of NAFLD are 1- simple fatty liver (steatosis); 2- non-alcoholic steatohepatitis (NASH), a more serious form of NAFLD; 3- fibrosis, where there is a persistent inflammation in the liver resulting in the generation of fibrous scar tissue around the liver cells and blood vessels; and 4-cirrhosis; this damage is permanent and can lead to liver failure and liver cancer.

NASH includes fat accumulation in the liver, as well as inflammation which over time can lead to increasing fibrosis, cirrhosis and end stage liver disease. Liver transplantation is the only treatment for advanced cirrhosis with liver failure, and transplantation is increasingly performed in persons suffering from NASH.

Estimates of the worldwide prevalence of NAFLD range from 6.3% to 33% with a median of 20% in the general population. The estimated prevalence of NASH is lower, ranging from 3 to 5% (Younossi et al., Hepatology, Vol. 64, No. 1, 2016). NASH is a worldwide problem with growing prevalence over the last few decades. Over the last decade NASH has risen from uncommon to the second indication for liver transplantation in the US. It is expected to be the leading cause of transplant by 2020 (Wong, et al, Gastro 2015). NASH is highly associated with the metabolic syndrome and Type 2 diabetes mellitus. NASH is a cause of progressive fibrosis and of cirrhosis. Cirrhosis due to NASH increases the risk of hepatocellular carcinoma and hepatocellular cancer. Furthremore, cardiovascular mortality is an important cause of death in NASH patients.

Chronic cholestasis and liver inflammation are the two main pathophysiological components of the two major classes of disease—primary biliary cirrhosis (PBC) and primary sclerosing cholangitis (PSC)—leading to bile duct destruction and ultimately to cirrhosis and liver failure. Liver transplantation appears to be the only life-saving procedure.

Ursodeoxycholic acid (UCDA), also known as ursodiol, is the main treatment for PBC. UCDA is a secondary bile acid, i.e. it is metabolized from a primary bile acid (produced by the liver) by intestinal bacteria, after the primary acid has been secreted into the intestine. UDCA is not an FXR agonist.

UDCA halts progression in many patients, but in about 30-40% of the population do not respond. Since May 2016, another molecule has been approved in the US for the treatment of PBC, when combined with UDCA for primary biliary cholangitis (PBC) in adult patients with an inadequate response to UDCA, or as a single therapy in adults unable to tolerate UDCA. This new molecule is Obeticholic acid (OCA), a bile-acid mimetic. OCA is a FXR agonist.

Currently there is no approved therapy for NASH.

There remains a need for efficacious treatments and therapies for liver conditions mediated by FXR, in particular liver diseases such as NAFLD, NASH or PBC, and for late stage liver diseases.

Development of NASH, involves several mechanisms: accumulation of fat in the liver (steatosis), inflammation of the liver, hepatocyte ballooning, and fibrosis. The NAFLD Activity Score (NAS) was developed as a tool to measure changes in NAFLD during therapeutic trials. The score is calculated as the unweighted sum of the scores for steatosis (0-3), lobular inflammation (0-3), and ballooning (0-2).

For preventing or treating such diseases or disorders, a medicament would be particularly efficient if it has an impact on each of these different aspects.

In a clinical trial aimed at evaluating the efficacy and safety of cenicriviroc for the treatment of NASH in adults with liver fibrosis, the treatment with CVC shown a reduction of fibrosis but no significant impact on the improvement of NAS. Furthermore CVC can induce fatigue or diarrhea in a small part of the patients (see “Tobira Therapeutics Announces Clinically and Statistically Significant Improvement in Liver Fibrosis from Phase 2b CENTAUR NASH Trial at One Year” Jul. 25, 2016).

When tested in nonalcoholic steatohepatitis patients, obeticholic acid showed efficacy, in particular a significant improvement in NAS, i.e. strong impact on steatosis with additional effects on inflammation and ballooning. But OCA long term administration raises safety concerns because it can be associated with pruritus, as well as with increased LDL cholesterol (see “Intercept Announces New FLINT Trial Data Showing OCA Treatment Increases Fibrosis Resolution and Cirrhosis Prevention in High-Risk NASH Patients”, Apr. 23, 2015). To avoid the risk of adverse cardiovascular events, concomitant administration of statins may be required for long term treatment of NASH patients.

Therefore there is a need to provide treatments for fibrotic / cirrhotic diseases or disorders, e.g. liver diseases or disorders, that can address the different aspects of these complex conditions, while demonstrating an acceptable safety and/or tolerability profile. The combination of two or more molecules with different Mechanisms of Action (MoA) might provide additional benefits for improving treatment efficacy and response rates.

### SUMMARY OF THE INVENTION

The invention provides pharmaceutical combinations, containing, separate or together, a FXR agonist and one or more additional therapeutic agent, for simultaneous, sequentially or separate administration. There is also provided a medicament, comprising such combinations.

According to the invention, the FXR agonist is a non-steroidal FXR agonist, and/or is a non-bile acid derived FXR agonist, e.g. is a non-bile acid derived FXR agonist.

In some aspects of the invention, the FXR agonist is 2-[3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid (Compound A), 4-((N-benzyl-8-chloro-1-methyl-1,4-dihydrochromeno[4,3-c]pyrazole-3 carboxamido)methyl)benzoic acid (Compound B), a pharmaceutically acceptable salt, solvate, prodrug, ester and/or an amino acid conjugate thereof.

In some aspects, the additional therapeutic agent is a CCR2/5 inhibitor, e.g. CVC, a pharmaceutically acceptable salt, solvate, prodrug and/or ester thereof, e.g. CVC, e.g. cenicriviroc mesylate.

There is also provided pharmaceutical combinations containing, separately or together, (i) a FXR agonist, e.g. a non-steroidal FXR agonist, and (ii) an additional therapeutic agent, e.g. a

CCR2/5 inhibitor, e.g. cenicriviroc, or a pharmaceutically acceptable salt, prodrug or solvate thereof, e.g. cenicriviroc mesylate, for simultaneous, sequential or separate administration.

Components (i) and (ii) can be administered together, one after the other or separately in one combined unit dose form or in two separate unit dose forms. The unit dose form may also be a fixed combination.

In some aspects, the pharmaceutical combination is a fixed combination, e.g. a fixed combination comprising (i) a FXR agonist, e.g. a non-steroidal FXR agonist, and (ii) an additional therapeutic agent, e.g. a CCR2/5 inhibitor, e.g. cenicriviroc (as herein defined, e.g. in free form or as a pharmaceutically acceptable salt thereof, e.g. cenicriviroc mesylate).

In some aspects, the FXR agonist and the additional therapeutic agent are provided for the treatment of a fibrotic disease or disorder, e.g. a liver disease or disorder, e.g. a chronic liver disease or disorder, e.g a disease or disorder selected from the group consisting of cholestasis, intrahepatic cholestasis, estrogen-induced cholestasis, drug-induced cholestasis, cholestasis of pregnancy, parenteral nutrition-associated cholestasis, primary biliary cirrhosis (PBC), primary sclerosing cholangitis (PSC), progressive familiar cholestasis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, dyslipidemia, atherosclerosis, diabetes, diabetic nephropathy, colitis, newborn jaundice, prevention of kernicterus, veno-occlusive disease, portal hypertension, metabolic syndrome, hypercholesterolemia, intestinal bacterial overgrowth, erectile dysfunction, progressive fibrosis of the liver caused by any of the diseases above or by infectious hepatitis, e.g.NAFLD, NASH, hepatic fibrosis, hepatosteatis or PBC.

In other aspects of the invention, the FXR agonist and the additional therapeutic agent are provided for slowing, arresting, or reducing the development of a cirrhotic disease or disorder, e.g. a chronic liver disease or disorder, e.g. NAFLD, NASH, liver fibrosis and PBC.

In yet another aspect, the FXR agonist and the additional therapeutic agent are provided for preventing or delaying progression of a chronic liver disease or disorder to a more advanced stage or a more serious condition thereof, e.g for preventing or delaying progression of a chronic liver disease or disorder selected from the group consisting of NAFLD, NASH, hepatic fibrosis and PBC.

In some aspects, the FXR agonist is 2-[3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid (Compound A), a stereoisomer, an enantiomer, a pharmaceutically acceptable salt, solvate, prodrug, ester thereof and/or an amino acid conjugate thereof.

In other aspects, the FXR agonist is 4-((N-benzyl-8-chloro-1-methyl-1,4-dihydrochromeno[4,3-c]pyrazole-3 carboxamido)methyl)benzoic acid (Compound B) a pharmaceutically acceptable salt, solvate, prodrug, ester thereof and/or an amino acid conjugate thereof.

The invention is also directed to pharmaceutical combinations comprising (i) a FXR agonist, e.g. a non-steroidal FXR agonist (e.g. Compound A, as herein defined, e.g. in free form or as a pharmaceutically acceptable salt or solvate thereof); or Compound B (as herein defined, e.g. in free form or as a pharmaceutically acceptable salt or solvate thereof), and (ii) a CCR2/5 inhibitor, e.g. cenicriviroc (as herein above defined, e.g. in free form or as a pharmaceutically acceptable salt or solvate thereof), optionally in the presence of a pharmaceutically acceptable carrier.

For example, there is provided pharmaceutical combinations comprising (i) a non-steroidal FXR agonist, e.g. Compound A, Compound B, a pharmaceutically acceptable salt, solvate, prodrug, ester and/or an amino acid conjugate thereof, and (ii) cenicriviroc, in free form or a pharmaceutically acceptable salt, solvate, prodrug and/or ester thereof, and (iii) a pharmaceutically acceptable carrier. In some embodiments of the invention, such a pharmaceutical combination is combined unit dose form.

In some aspects, there is provided pharmaceutical combinations comprising (i) a non-steroidal FXR agonist, and (ii) at least one additional therapeutic agent, e.g. cenicriviroc, a pharmaceutically acceptable salt, solvate, prodrug and/or ester thereof, in a quantity which is jointly therapeutically effective for use in the treatment or prevention of fibrotic or cirrhotic diseases or disorders, e.g. liver diseases or disorders, e.g. NAFLD, NASH, liver fibrosis or PBC.

Furthermore, the invention relates to such pharmaceutical combinations, e.g. fixed or free combinations, e.g. combined unit doses, for use in treating, preventing or ameliorating a fibrotic or cirrhotic disease or disorder, e.g. a liver disease or disorder. In some aspects, such methods comprise administering to a subject in need thereof the FXR agonist and the additional therapeutic agent, e.g. a CCR2/5 inhibitor, e.g. cenicriviroc (in free form or as a pharmaceutically acceptable salt, solvate, prodrug, and/or ester thereof, e.g. cenicriviroc mesylate), each being in an amount that is jointly therapeutically effective.

There is provided the use of a non-bile acid derived FXR agonist in combination, e.g. fixed or free combination, with one or more additional therapeutic agent, e.g. a CCR2/5 inhibitor e.g. cenicriviroc (or a pharmaceutically acceptable salt, solvate, prodrug and/or ester thereof, e.g. cenicriviroc mesylate), for the manufacture of a medicament for the prevention or treatment of a liver disease or disorder, e.g. a liver disease or disorder selected from the group consisting of NAFLD, NASH, hepatosteatosis, liver fibrosis, cirrhosis, PBC.

There is also provided pharmaceutical combinations for use preventing, delaying or treating a liver disease or disorder, wherein the combination comprises (i) a non-bile acid derived FXR agonist (e.g. Compound A, Compound B, as herein defined (e.g. in free form, or a pharmaceutically acceptable salt or solvate thereof), and (ii) a CCR2/5 inhibitor e.g. cenicriviroc (in free form or as a pharmaceutically acceptable salt, solvate, prodrug and/or ester thereof, e.g. cenicriviroc mesylate).

In some aspects of the invention, there is provided pharmaceutical combinations for use in preventing, delaying or treating a chronic liver disease or disorder, e.g. selected from the group consisting of steatosis, NASH, fibrosis and cirrhosis, e.g. steatosis, NASH and/or fibrosis, wherein the combination comprises (i) a non-bile acid derived FXR agonist (e.g. Compound A, Compound B, as herein defined, e.g. in free form, or a pharmaceutically acceptable salt or solvate thereof), and (ii) a CCR2/5 inhibitor e.g. cenicriviroc (in free form or as a pharmaceutically acceptable salt, solvate, prodrug and/or ester thereof, e.g. in free form or as a pharmaceutically acceptable salt thereof, e.g. cenicriviroc mesylate).

There is further provided pharmaceutical combinations comprising (i) a non-bile acid derived FXR agonist (e.g. Compound A or Compound B, as herein defined, e.g. in free form or a pharmaceutically acceptable salt or solvate thereof), and (ii) a CCR2/5 inhibitor e.g. cenicriviroc (in free form or as a pharmaceutically acceptable salt, solvate, prodrug, and/or ester thereof, e.g. in free form or as a pharmaceutically acceptable salt thereof, e.g. cenicriviroc mesylate), for use preventing, delaying or treating NASH.

Furthermore, there is also provided pharmaceutical combinations comprising (i) a non-bile acid derived FXR agonist (e.g. Compound A or Compound B, as herein defined, e.g. in free form or a pharmaceutically acceptable salt or solvate thereof), and (ii) a CCR2/5 inhibitor e.g. cenicriviroc (in free form or as a pharmaceutically acceptable salt, solvate, prodrug, and/or ester thereof, e.g. in free form or as a pharmaceutically acceptable salt thereof, e.g. cenicriviroc mesylate), for use preventing, delaying or treating liver fibrosis.

There is also provided pharmaceutical combinations comprising (i) a non-bile acid derived FXR agonist (e.g. Compound A or Compound B, as herein defined, e.g. in free form or as a pharmaceutically acceptable salt thereof), and (ii) a CCR2/5 inhibitor e.g. cenicriviroc (in free form or as a pharmaceutically acceptable salt, solvate, prodrug, and/or ester thereof, e.g. in free form or as a pharmaceutically acceptable salt thereof, e.g. cenicriviroc mesylate), for use in preventing, delaying or treating hepatosteatosis.

There is further provided pharmaceutical combinations comprising (i) a non-bile acid derived FXR agonist (e.g. Compound A or Compound B, as herein defined, e.g. in free form or as a pharmaceutically acceptable salt thereof), and (ii) a CCR2/5 inhibitor e.g. cenicriviroc (in free form or as a pharmaceutically acceptable salt, solvate, prodrug, and/or ester thereof, e.g. in free form or as a pharmaceutically acceptable salt thereof e.g. cenicriviroc mesylate), for use in preventing, delaying or treating hepatocellular ballooning.

There is also provided pharmaceutical combinations comprising (i) a non-bile acid derived FXR agonist (e.g. Compound A or Compound B, as herein defined, e.g. in free form or as a pharmaceutically acceptable salt thereof), and (ii) a CCR2/5 inhibitor e.g. cenicriviroc (in free form or as a pharmaceutically acceptable salt, solvate, prodrug, and/or ester thereof, e.g. in free form or as a pharmaceutically acceptable salt thereof, e.g. cenicriviroc mesylate), for use in preventing, delaying or treating PBC.

A further aspect of the present invention is a method for the treatment, delaying or prevention of a fibrotic disease or disorder, e.g. a liver disease or disorder, e.g. chronic liver disease or disorder, comprising administering a therapeutically effective amount of combination of (i) a non-bile acid derived FXR agonist, e.g. Compound A or Compound B as herein above defined (e.g. in free form or as a pharmaceutically acceptable salt thereof), and (ii) an additional therapeutic agent, as hereindefined, e.g. a CCR2/5, e.g. cenicriviroc (in free form or as a pharmaceutically acceptable salt, solvate, prodrug, and/or ester thereof, e.g. in free form or as a pharmaceutically acceptable salt thereof), and a pharmaceutically acceptable carrier to a subject in need of such treatment. A therapeutically effective amount of each of the component of the combination of the present invention may be administered simultaneously or sequentially and in any order.

In other embodiments the additional therapeutic agent is a CCR2/5 inhibitor, e.g cenicriviroc (in free form or as a pharmaceutically acceptable salt, solvate, prodrug, and/or ester thereof, e.g. in free form or as a pharmaceutically acceptable salt thereof). In some embodiments, the new dosing regimens are provided for use in preventing, delaying or treating a fibrotic or cirrhotic disease or disorder, e.g. a liver disease or disorder, e.g. a chronic liver disease or disorder, e.g. selected from the group consisting of NAFLD, NASH, liver fibrosis, cirrhosis and PBC, e.g NASH, liver fibrosis or PBC. In some embodiments, the new dosing regimens are provided for preventing, delaying or treating renal fibrosis.

There is also provided pharmaceutical combinations containing, separate or together, (i) Compound A as herein defined (e.g. in free form or a pharmaceutically acceptable salt thereof); and (ii) a CCR2/5 inhibitor, e.g. cenicriviroc (as herein defined, e.g. in free form or as a pharmaceutically acceptable salt thereof), e.g. for simultaneous or sequential administration, wherein the ratio (μg/mg (microgram/milligram)) of Compound A to CCR2/5 inhibitor is from about 3:100 to about 100:100; e.g. from about 5:100 to about 40:100; e.g. about 3:100, e.g. about 60:100. In particular there are provided pharmaceutical combinations containing, separate or together, (i) Compound A in free form or pharmaceutically acceptable salt or solvate thereof and cenicriviroc (as hereinavove defined), e.g. cenicriviroc mesylate, in particular containing Compound A, wherein the ratio (μg/mg (microgram/milligram)) of Compound A to cenicriviroc is from about 3:100 to about 100:100; e.g. from about 5:100 to about 40:100; e.g. about 3:100, e.g. about 60:100.

In other embodiments, there are provided pharmaceutical combinations, containing, separate or together, (i) Compound B as herein defined, e.g. in free form or a pharmaceutically acceptable salt thereof; and (ii) a CCR2/5 inhibitor, e.g. cenicriviroc (as hereinabove defined, e.g. in free form or as a pharmaceutically acceptable salt thereof), for simultaneous or sequential administration, wherein the ratio (mg/mg) of Compound B to CCR2/5 inhibitor, e.g. cenicriviroc (as hereinabove defined), is about 0.5:1 to about 10:1, e.g. about 0.5:1 to about 8:1, e.g. about 0.5:1 to about 5:1; about 0.5:1 to about 3:1, e.g. about 1:1 to about 5:1, e.g. about 1:1 to about 3:1, e.g. about 1:1 to about 2:1, e.g. about 1:1. In particular there are provided pharmaceutical combinations containing, separate or together, (i) Compound A as herein defined, e.g. in free form or a pharmaceutically acceptable salt thereof, and cenicriviroc (as hereinavove defined, e.g. in free form or as a pharmaceutically acceptable salt thereof, e.g. cenicriviroc mesylate, in particular containing Compound A, wherein the ratio (μg/mg (microgram/milligram)) of Compound A to cenicriviroc is from about 0.5:1 to about 10:1, e.g. about 0.5:1 to about 8:1, e.g. about 0.5:1 to about 5:1; about 0.5:1 to about 3:1, e.g. about 1:1 to about 5:1, e.g. about 1:1 to about 3:1, e.g. about 1:1 to about 2:1, e.g. about 1:1.

Various (enumerated) embodiments of the invention are described herein. It will be recognized that features specified in each embodiment may be combined with other specified features to provide further embodiments of the present invention.

### DESCRIPTION OF THE FIGURES

FIGS. 1.1 to 1.3 describe an in-vivo efficacy study of cenicriviroc in combination with Compound A (listed as NVP-CH-4) and Compound B (listed as NVP-CH-5) in the STAM model of non-alcoholic steatohepatitis fora treatment period of 6-9 and 9-12 weeks. FIG. 1.1 refers to the results on NAFLD Activity Score; FIG. 1.2. to the Inflammatory Cell Infiltration Score and FIG. 1.3 to the heaptocyte Ballooning Score.

FIG. 2 describes an in-vivo efficacy study of cenicriviroc in combination with Compound A (listed as NVP-CH-4) and Compound B (listed as NVP-CH-5) in the STAM model of non-alcoholic steatohepatitis.

### DETAILED DESCRIPTION OF THE INVENTION

Definitions

For purposes of interpreting this specification, the following definitions will apply and whenever appropriate, terms used in the singular will also include the plural and vice versa.

As used herein, the term “about” in relation to a numerical value x means +/-10%, unless the context dictates otherwise.

As used herein, the term “amino acid conjugate” refers to conjugates of Compound A or Compound B with any suitable amino acid. Preferably, such suitable amino acid conjugates of Compound A or Compound B will have the added advantage of enhanced integrity in bile or intestinal fluids. Suitable amino acids include but are not limited to glycine, taurine and acylglucuronide. Thus, the present invention encompasses the glycine, taurine and acylglucuronide conjugates of Compound A or Compound B.

As used herein, the term “FXR agonist” refers to an agent that directly binds to and upregulates the activity of FXR.

As used herein, the terms “salt” or “salts” refers to an acid addition or base addition salt of a compound of the invention. “Salts” include in particular “pharmaceutical acceptable salts”.

As used herein, the term “pharmaceutically acceptable” means a nontoxic material that does not interfere with the effectiveness of the biological activity of the active ingredient(s).

As used herein, the term “amino acid conjugate” refers to conjugates of the compounds, e.g. of Compound A or Compound B, with any suitable amino acid. Preferably, such suitable amino acid conjugates of Compound A or Compound B will have the added advantage of enhanced integrity in bile or intestinal fluids. Suitable amino acids include but are not limited to glycine, taurine and acyl glucuronide. Thus, the present invention encompasses the glycine, taurine and acyl glucuronide conjugates Compound A or Compound B.

As used herein the term “prodrug” refers to compound that is converted in vivo to the compounds of the present invention. A prodrug is active or inactive. It is modified chemically through in vivo physiological action, such as hydrolysis, metabolism and the like, into a compound of this invention following administration of the prodrug to a subject. The suitability and techniques involved in making and using pro-drugs are well known by those skilled in the art. Suitable prodrugs are often pharmaceutically acceptable ester derivatives.

As used herein, the terms “patient” or “subject” refer to a human.

As used herein, the term “treat”, “treating” or “treatment” of any disease or disorder refers in one embodiment to ameliorating the disease or disorder (i.e. slowing or arresting or reducing the development of the disease or at least one of the clinical symptoms or pathological features thereof). In another embodiment “treat”, “treating” or “treatment” refers to alleviating or ameliorating at least one physical parameter or pathological features of the disease, e.g. including those which may not be discernible by the subject. In yet another embodiment, “treat”, “treating” or “treatment” refers to modulating the disease or disorder, either physically, (e.g. stabilization of at least one discernible or non-discernible symptom), physiologically (e.g. stabilization of a physical parameter) or both. In yet another embodiment, “treat”, “treating” or “treatment” refers to preventing or delaying the onset or development or progression of the disease or disorder, or of at least one symptoms or pathological features associated thereof. In yet another embodiment, “treat”, “treating” or “treatment” refers to preventing or delaying progression of the disease to a more advanced stage or a more serious condition, such as e.g. liver cirrhosis; or to preventing or delaying a need for liver transplantation.

For example, treating NASH may refer to ameliorating, alleviating or modulating at least one of the symptoms or pathological features associated with NASH; e.g. hepatosteatosis, hepatocellular ballooning, hepatic inflammation and fibrosis; e.g. may refer to slowing progression, reducing or stopping at least one of the symptoms or pathological features associated with NASH, e.g. hepatosteatosis, hepatocellular ballooning, hepatic inflammation and fibrosis. It may also refer to preventing or delaying liver cirrhosis or a need for liver transplantation.

As used herein, the term “therapeutically effective amount” refers to an amount of the compound of the invention, e.g. FXR agonist, e.g. Compound A or Compound B (as hereinabove defined), which is sufficient to achieve the stated effect. Accordingly, a therapeutically effective amount of a FXR agonist, e.g. Compound A or Compound B (as hereinabove defined), used for the treatment or prevention of a liver disease or disorder as hereinabove defined is an amount sufficient for the treatment or prevention of such a disease or disorder.

By “therapeutic regimen” is meant the pattern of treatment of an illness, e.g., the pattern of dosing used during the treatment of the disease or disorder.

As used herein, a subject is “in need of” a treatment if such subject would benefit biologically, medically or in quality of life from such treatment.

As used herein, the term “liver disease or disorder” encompasses one, a plurality, or all of non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis and liver fibrosis.

As used herein, the term NAFLD may encompass the different stages of the disease: hepatosteatosis, NASH, fibrosis and cirrhosis.

As used herein, the term NASH may encompass steatosis, hepatocellular ballooning and lobular inflammation.

As herein defined, “combination” refers to either a fixed combination in one unit dosage form (e.g., capsule, tablet, or sachet), free (i.e. non-fixed) combination, or a kit of parts for the combined administration where a FXR agonist of the present invention and one or more “combination partner” (i.e. the additional therapeutic agent, such as e.g. cenicriviroc or a pharmaceutically acceptable salt or solvate thereof, also referred to as or “co-agent”) may be administered independently at the same time or separately within time intervals, especially where these time intervals allow that the combination partners show a cooperative, e.g. synergistic effect.

The terms “co-administration” or “combined administration” or the like as utilized herein are meant to encompass administration of the additional therapeutic agent to a single subject in need thereof (e.g. a patient), and the additional therapeutic agent are intended to include treatment regimens in which the FXR agonist and the additional therapeutic agent are not necessarily administered by the same route of administration and/or at the same time. Each of the components of the combination of the present invention may be administered simultaneously or sequentially and in any order. Co-administration comprises simultaneous, sequential, overlapping, interval, continuous administrations and any combination thereof.

The term “pharmaceutical combination” as used herein means a pharmaceutical composition that results from the combining (e.g. mixing) of more than one active ingredient and includes both fixed and free combinations of the active ingredients.

The term “fixed combination” means that the active ingredients, i.e. i) a non-bile acid derived FXR agonist, e.g. Compound A or Compound B (in free form or e.g. as a pharmaceutically acceptable salt or an amino acid conjugate thereof) and ii) the additional therapeutic agent, e.g.

cenicriviroc (as herein defined, e.g. cenicriviroc mesylate), are both administered to a patient simultaneously in the form of a single entity or dosage.

The term “free combination” means that the active ingredients as hereindefined are both administered to a patient as separate entities either simultaneously, concurrently or sequentially with no specific time limits, and in any order, wherein such administration provides therapeutically effective levels of the two compounds in the body of the patient.

By “simultaneous administration”, it is meant that the FXR agonist and the additional therapeutic agent, e.g. cenicriviroc (as herein defined, e.g. cenicriviroc mesylate), are administered on the same day. The two active ingredients can be administered at the same time (for fixed or free combinations) or one at a time (for free combinations).

According to the invention, “sequential administration”, may mean that during a period of two or more days of continuous co-administration only one of the FXR agonist and the additional therapeutic agent, e.g. cenicriviroc (as herein defined, e.g. cenicriviroc mesylate), is administered on any given day.

By “overlapping administration”, it is meant that during a period of two or more days of continuous co-administration, there is at least one day of simultaneous administration and at least one day when only one of FXR agonist and the additional therapeutic agent, e.g. cenicriviroc (as herein defined, e.g. cenicriviroc mesylate), is administered.

By “interval administration”, it is meant a period of co-administration with at least one void day, i.e with at least one day where neither the FXR agonist nor the additional therapeutic agent, e.g. cenicriviroc (as herein defined, e.g. cenicriviroc mesylate, is administered.

By “continuous administration”, it is meant a period of co-administration without any void day. The continuous administration may be simultaneous, sequential, or overlapping, as described above.

FXR Agonists

According to the invention, the FXR agonist can be selected from the group consisting of Compound A (as hereinabove defined, e.g. including stereoisomer, enantiomer, pharmaceutically acceptable salt, solvate, prodrug, ester and amino acid conjugate thereof), Compound B (as hereinabove defined, e.g. including pharmaceutically acceptable salt, solvate prodrug, ester and amino acid conjugate thereof), GS-9676, GS-9674 (both non-bile acid derived FXR agonists, from Gilead, or a pharmaceutically acceptable salt thereof), PX102/104.

In one embodiment of the invention, the FXR agonist can be a non-bile acid derived FXR agonist, e.g. a non-steroidal FXR agonist. E.g. can be selected from the group consisting of Compound A (as hereinabove defined, e.g. in free form or a pharmaceutically acceptable salt thereof), Compound B (as hereinabove defined, e.g. in free form or a pharmaceutically acceptable salt thereof, e.g. meglumine salt), GS-9676, and a mixture thereof.

Compound A is meant for 2-[3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid. Compound A can be in free form or as a pharmaceutically acceptable salt or an amino acid conjugate thereof; e.g. glycine conjugate, taurine conjugate or acyl glucuronide conjugate. Compound A can also encompass a stereoisomer, an enantiomer thereof. Compound A can also be administered as a prodrug, an ester, in form of a polymorph, solvate and/or hydrate.

Compound B is 4-((N-benzyl-8-chloro-1-methyl-1,4-dihydrochromeno[4,3-c]pyrazole-3 carboxamido)methyl)benzoic acid. Compound B can be in free form or as a pharmaceutically acceptable salt, solvate, prodrug, ester and/or an amino acid conjugate thereof.

Compound B can be 4-((N-benzyl-8-chloro-1-methyl-1,4-dihydrochromeno[4,3-c]pyrazole-3 carboxamido)methyl)benzoic acid meglumine salt. In one embodiment, Compound B is 4-((N-benzyl-8-chloro-1-methyl-1,4-dihydrochromeno[4,3-c]pyrazole-3 carboxamido)methyl)benzoic acid meglumine salt Form A or Form B. In another embodiment, Compound B is 4-((N-benzyl-8-chloro-1-methyl-1,4-dihydrochromeno[4,3-c]pyrazole-3 carboxamido)methyl)benzoic acid meglumine mono-hydrate. In yet another embodiment, Compound B is 4-((N-benzyl-8-chloro-1-methyl-1,4-dihydrochromeno[4,3-c]pyrazole-3 carboxamido)methyl)benzoic acid meglumine mono-hydrate Form H_{A} or mono-hydrate Form H_{B}.

Any formula given herein is also intended to represent unlabeled forms as well as isotopically labeled forms of the compounds.

Combination Partners

According to the invention, the combination partner of the invention can be a CCR2/5 inhibitor, e.g. cenicriviroc (in free form or as a pharmaceutically acceptable salt, solvate, prodrug and/or ester thereof, e.g. cenicriviroc mesylate).

Another CC2/5 inhibitor can be (S)-1-[(1S,2R,4R)-4-isopropyl(methyl)amino)-2-propylcyclohexyl]-3-(6-(trifluoromethyl)quinazolin-4-ylamino)pyrrolid in-2-one, e.g as described in WO2011/046916.

Another CC2/5 inhibitor can be BMS-813160.

Modes of Administration

The pharmaceutical composition of the invention can be formulated to be compatible with its intended route of administration (e.g. oral compositions generally include an inert diluent or an edible carrier). Other non-limiting examples of routes of administration include parenteral (e.g. intravenous), intradermal, subcutaneous, oral (e.g. inhalation), transdermal (topical), transmucosal, and rectal administration. The pharmaceutical compositions compatible with each intended route are well known in the art.

Diseases

As hereinabove defined, the fibrotic or cirrhotic disease or disorder can be a liver disease or disorder, e.g. as defined below herein, or renal fibrosis.

As hereinabove defined, the liver diseases or disorders can be cholestasis, intrahepatic cholestasis, estrogen-induced cholestasis, drug-induced cholestasis, cholestasis of pregnancy, parenteral nutrition-associated cholestasis, primary biliary cirrhosis (PBC), primary sclerosing cholangitis (PSC), progressive familiar cholestasis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, dyslipidemia, atherosclerosis, diabetes, diabetic nephropathy, colitis, newborn jaundice, prevention of kernicterus, veno-occlusive disease, portal hypertension, metabolic syndrome, hypercholesterolemia, intestinal bacterial overgrowth, erectile dysfunction, progressive fibrosis of the liver caused by any of the diseases above or by infectious hepatitis.

The liver diseases or disorders can also refer to liver transplantation.

In one embodiment of the invention, the pharmaceutical combination (as herein defined) is for the treatment or prevention of a fibrotic disease or disorder, e.g. a liver disease or disorder, e.g. a chronic liver disease, e.g. a liver disease or disorder selected from the group consisting of PBC, NAFLD, NASH, drug-induced bile duct injury, gallstones, liver cirrhosis, alcohol-induced cirrhosis, cystic fibrosis-associated liver disease (CFLD), bile duct obstruction, cholelithiasis, liver fibrosis. In one embodiment of the invention, the pharmaceutical combination (as herein defined) is for the treatment or prevention of fibrosis, e.g. renal fibrosis or liver fibrosis.

According to one embodiment of the invention, the liver diseases or disorders refer to NAFLD, e.g. any stages of NAFLD, e.g. any of steatosis, NASH, fibrosis and cirrhosis.

In one embodiment of the invention, there is provided a pharmaceutical combination of the invention for the improvement of liver fibrosis without worsening of steatohepatitis

In another embodiment of the invention, there is provided a pharmaceutical combination of the invention for obtaining a complete resolution of steatohepatitis without worsening, e.g. improving, of liver fibrosis.

In another embodiment of the invention, there is provided a pharmaceutical combination of the invention for preventing or treating steatohepatitis and liver fibrosis.

In yet another embodiment of the invention, there is provided a pharmaceutical combination of the invention for reducing at least one of the features of the NAS score, i.e. one of hepatosteatosis, hepatic inflammation and hepatocellular ballooning; e.g. at least two features of the NAS score, e.g. hepatosteatosis and hepatic inflammation, or hepatosteatosis and hepatocellular ballooning, or hepatocellular ballooning and hepatic inflammation.

In a further embodiment of the invention, there is provided a pharmaceutical combination of the invention for reducing at least one or two features of the NAS score and liver fibrosis, e.g. for reducing hepatic inflammation and liver fibrosis, or hepatosteatosis and liver fibrosis or hepatocellular ballooning and liver fibrosis.

In yet a further embodiment of the invention there is provided a pharmaceutical combination for treating or preventing, stage 3 fibrosis to stage 1 fibrosis, e.g. stage 3 and/or stage 2 and/or stage 1 fibrosis.

Patients

According to the invention, the patients receiving the combination of the invention can be affected or at risk of a fibrotic disease or disorder, e.g. a liver disease or disorder, e.g. as hereinabove defined.

In some embodiments of the invention, the patient is obese or overweight

In other embodiments of the invention, the patient may be a diabetic patient, e.g. may have type 2 diabetes. The patient may have high blood pressure and/or high blood cholesterol level.

Dosing Regimens

Depending on the patient general condition, the targeted disease or disorder and the stage of such disease or disorder, the dosing regimen, i.e. administered doses and/or frequency of each component of the pharmaceutical combination may vary.

The frequency of dosing of the FXR agonist of the invention and the additional therapeutic agent, e.g as a fixed dose combination, may be once per day, twice per day, three times per day, four times per day, five times per day, six times per day, or every two days, every three days or once per week, e.g. once a day.

According to the invention, the FXR agonist and the additional therapeutic agent may not be administered following the same regimen, i.e. may not be administered at the same frequency and/or duration and/or dosage, e.g. at the same frequency and/or dosage. This can be the case e.g. for free combinations. As one example, the FXR agonist can be administered one a day and the additional therapeutic agent, e.g. a CCR2/5 inhibitor, e.g. cenicriviroc (in free form or as a pharmaceutically acceptable salt, solvate, prodrug and/or ester thereof, e.g. cenicriviroc mesylate) twice per day, or reciprocally.

In one embodiment, e.g. in case of simultaneous administration, the FXR agonist is administered one to four times per day, and the additional therapeutic agent, e.g. cenicriviroc (in free form or as a pharmaceutically acceptable salt, solvate, prodrug and/or ester thereof) is administered from one to four times per day.

In one embodiment of the invention, the co-administration is carried out for at least one week, at least one month, at least 6 weeks, at least three months, at least 6 months, at least one year. For example, the pharmaceutical combination of the invention is administered lifelong to the patient. The frequency of administration, and/or the doses of the FXR agonist and of the additional therapeutic agent, may vary during the whole period of administration.

During the treatment, there can be one or more periods of time, e.g. days, during which nor the FXR agonist of the invention neither the additional therapeutic agent, e.g. a CCR2/5 inhibitor, e.g. cenicriviroc (in free form or as a pharmaceutically acceptable salt, solvate, prodrug and/or ester, e.g. cenicriviroc mesylate) are administered to the patient (i.e. periods, e.g. days, void of combination treatment), or during which only one drug amongst the FXR agonist or the additional therapeutic agent is administered to the patient.

In case of a sequential co-administration, the FXR agonist may be administered prior the additional therapeutic agent, or reciprocally. The time interval between administration of the FXR agonist and of the additional therapeutic agent may vary from a few minutes to a few days, e.g. a few minutes, e.g. a few hours, e.g. 1 day to 1 week.

The dosing frequency will depend on, inter alia, the phase of the treatment regimen.

According to the invention, the non-bile acid derived FXR agonist, e.g. Compound A (as hereinabove defined, e.g. in free form or as a pharmaceutically acceptable salt thereof), is administered ata dose of about 3 μgto about 100 μg, e.g. about 5 μg to about 100 μg, e.g. about 10 μg to about 100 μg, e.g. about 20 μg to 100 μg delivered orally, e.g. about 30 μg to about 90 μg, e.g. about 40 μg to about 60 μg. Such doses amay be for oral administration. Such doses may be for daily administration, or twice daily administration or every two days administration, e.g. for daily oral administration, twice daily oral administration or every two days oral administration.

In some aspects, the non-bile acid derived FXR agonist e.g. Compound A (as herein above defined, e.g in free form or as a pharmaceutically acceptable salt thereof) that is administered with an additional therapeutic agent, e.g. cenicriviroc (in free form or as a pharmaceutically acceptable salt, solvate, prodrug and/or ester thereof, e.g. cenicriviroc mesylate), is administered at a dose of about 10 μg , about 25 μg, about 30 μg, about 60 μg or about 90 μg. Such doses may be for daily or twice daily, e.g. for daily administration. Such doses are particularly adapted for oral administration of the FXR agonist, e.g. Compound A (in free form or as a pharmaceutically acceptable salt thereof).

In some embodiments, the non-bile acid derived FXR agonist, e.g. Compound A as herein defined (e.g. in free form or a pharmaceutically acceptable salt thereof), is administered at a dose in a range of about 20 μg- about 60 μg delivered orally, e.g. about 30 μg- about 60 μg delivered orally. Such doses may be for daily administration (daily doses), or twice daily administration or every two days administration, e.g. for daily administration.

In some embodiments, the non bile acid derived FXR agonist, e.g. Compound A as herein defined (e.g. in free form or a pharmaceutically acceptable salt thereof), is administered at a dose of, about 10 μg to 60 μg delivered orally, e.g. about 10 μg to about 40 μg delivered orally, e.g. about 20 μg to about 40 μg delivered orally. Such doses may be for daily administration (daily doses), or twice daily administration or every two days administration, e.g. for daily administration.

In some embodiments, the non bile acid derived FXR agonist, e.g. Compound A as herein defined (e.g. in free form or a pharmaceutically acceptable salt thereof), is administered at a dose in a range of about 5 μg to about 60 μg delivered orally, e.g. about 5 μg to about 40 μg delivered orally. Such doses may be for daily administration (daily doses), or twice daily administration or every two days administration, e.g. for daily administration.

In other embodiments, the non bile acid derived FXR agonist, e.g. Compound A as herein defined (e.g. in free form or a pharmaceutically acceptable salt thereof), is administered at a dose in a range of about 3 μg to about 40 μg delivered orally, e.g. about 3 μg to about 30 μg delivered orally. Such doses may be for daily administration (daily doses), or twice daily administration or every two days administration, e.g. for daily administration.

In some embodiments, the non bile acid derived FXR agonist, e.g. Compound A as herein defined (e.g. in free form or a pharmaceutically acceptable salt thereof), is administered at a dose of about 3 μg delivered orally, about 4 μg delivered orally, about 5 μg delivered orally, about 10 μg delivered orally, about 20 μg delivered orally, about 25 μg delivered orally, about 30 μg delivered orally, about 40 μg delivered orally, about 60 μg delivered orally, or about 90 μg delivered orally. Such doses may be for oral administration.

In some embodiments, the non bile acid derived FXR agonist, e.g. Compound A as herein defined (e.g. in free form or a pharmaceutically acceptable salt thereof), is administered at a dose in a range of about 3 μg/day to about 100 μg/day, e.g. about 5 μg/day to about 100 μg/day, e.g. about 10 μg/day to about 100 μg/day, e.g. about 20 μg/day to 100 μg/day, e.g. about 30 μg/day to about 90 μg/day, e.g. about 40 μg/day to about 60 μg/day, e.g. about 10 μg/day to 60 μg/day, e.g. about 10 μg/day to about 40 μg/day, e.g. about 20 μg/day to 40 μg/day, e.g. about 20 μg/day to about 60 μg/day, e.g. about 30 μg/day to about 60 μg/day, e.g. about 5 μg/day to 60 μg/day, e.g. about 5 μg/day to 40 μg/day, e.g. about 3 μg/day to about 40 μg/day, about 3 μg/day to about 30 μg/day.

In some embodiments, the non bile acid derived FXR agonist, e.g. Compound A as herein defined (e.g. in free form or a pharmaceutically acceptable salt thereof), is administered at a dose of about 3 μg/day, about 4 μg/day, about 5 μg/day, about 10 μg/day, about 25 μg/day, about 30 μg/day, about 60 μg/day, or about 90 μg/day. Such regimens may be delivered orally.

In some embodiments, the non bile acid derived FXR agonist, e.g. Compound A as herein defined (e.g. in free form or a pharmaceutically acceptable salt thereof), is administered at a dose of about 3 μg twice daily, about 4 μg twice daily, about 5 μg twice daily, about 10 μg twice daily, about 25 μg twice daily, about 30 μg twice daily. Such regimens may be delivered orally.

In some embodiments, the non bile acid derived FXR agonist, e.g. Compound A as herein defined (e.g. in free form or a pharmaceutically acceptable salt thereof) is administered at a dose of about 5 μg every two days, about 10 μg every two days, about 40 μg every two days, about 60 μg every two days. Such regimens may be delivered orally.

Such doses and regimens are particulary adapted for Compound A in free form.

In some embodiments, the FXR agonist, e.g. non bile acid derived FXR agonist, e.g. Compound A as herein defined (e.g. in free form or a pharmaceutically acceptable salt thereof), is to be administered at a daily dose of about 3 μg or about 5 μg.

In some embodiments, the FXR agonist, e.g. non bile acid derived FXR agonist, e.g. Compound A as herein defined (e.g. in free form or a pharmaceutically acceptable salt thereof), is to be administered at a daily dose of about 10 μg.

In some embodiments, the FXR agonist, e.g. non bile acid derived FXR agonist, e.g. Compound A as herein defined (e.g. in free form or a pharmaceutically acceptable salt thereof) is to be administered at a daily dose of about 20 μg or 25 μg.

In some embodiments, the FXR agonist, e.g. non bile acid derived FXR agonist, e.g. Compound A as herein defined (e.g. in free form or a pharmaceutically acceptable salt thereof) is to be administered at a daily dose of about 30 μg.

In some embodiments, the FXR agonist, e.g. non bile acid derived FXR agonist, e.g. Compound A as herein defined (e.g. in free form or a pharmaceutically acceptable salt thereof) is to be administered at a daily dose of about 40 μg.

In some embodiments, the FXR agonist, e.g. non bile acid derived FXR agonist, e.g. Compound A as herein defined (e.g. in free form or a pharmaceutically acceptable salt thereof) is to be administered at a daily dose of about 60 μg.

In some embodiments, the FXR agonist, e.g. non bile acid derived FXR agonist, e.g. Compound A as herein defined (e.g. in free form or a pharmaceutically acceptable salt thereof), is administered in such a way to provide a Cₘₐₓ of the FXR agonist of at least about 0.2 ng/mL, e.g. in a range of about 0.2 to about 2.0 ng/mL, e.g. about 0.2 to about 1.0 ng/mL, e.g. about 0.2 to about 0.5 ng/mL.

Alternatively, the administered dose may be expressed in units of mg/m²/day in which a patient body surface area (BSA) may be calculated in m² using various available formulae using the patient height and weight. It is straightforward to convert from one unit to another given a patient's height and weight.

According to the invention, Compound B (as hereinabove defined, e.g. in free form or as a pharmaceutically acceptable salt thereof) is administered at a dose of about 50 mg, e.g. about 60 mg, e.g about 80 mg, e.g. about 100 mg, e.g. about 120 mg, e.g. about 140 mg, e.g. about 150 mg, e.g about 180 mg, e.g about 200 mg, e.g about 220 mg, e.g about 250 mg. Such doses may be for oral administration of Compound B. Such doses may be for daily administration of Compound B, twice daily administration or every two days administration, e.g. for daily oral administration.

In some aspects, the non-bile acid derived FXR agonist, e.g. Compound B (as herein above defined, e.g in free form or as a pharmaceutically acceptable salt thereof) is administered at a dose in a range of about 30 mg to about 250 mg, e.g about 50 mg to about 250 mg, e.g. about 100 mg to about 250 mg, e.g. about 10 mg to about 200 mg; e.g. about 100 mg to about 200 mg; e.g. about 30 mg to about 200 mg, e.g. about 50 mg to about 200 mg. Such doses may be for oral administration of Compound B. Such doses may be for daily administration of Compound B, twice daily administration or every two days administration, e.g. for daily oral administration. These doses can be in particular for meglumine salt of Compound B.

In some embodiments, the non-bile acid derived FXR agonist, e.g. Compound B as herein defined (e.g. in free form or a pharmaceutically acceptable salt thereof) is administered at a dose of about 50 mg delivered orally, about 60 mg delivered orally, about 80 mg delivered orally, about 100 mg delivered orally, about 120 mg delivered orally, about 140 mg delivered orally, about 150 mg delivered orally, about 180 mg delivered orally, about 200 mg delivered orally, about 220 mg delivered orally, about 250 mg delivered orally. Such doses may be particularly adapted for patients of weight from about 50 kg to about 120 kg, e.g. from about 70 kg to about 100 kg. These doses can be in particular for meglumine salt of Compound B.

In some embodiments, the non-bile acid derived FXR agonist, e.g. Compound B as herein defined (e.g. in free form or a pharmaceutically acceptable salt thereof) is administered at a dose in a range of about 50 mg/day, e.g. about 60 mg/day, e.g about 80 mg/day, e.g. about 100 mg/day, e.g. about 120 mg/day, e.g. about 140 mg/day, e.g. about 150 mg/day, e.g. about 180 mg/day, e.g. about 200 mg/day, e.g. about 220 mg/day, e.g. about 250 □g/day. Such regimens may be delivered orally. These doses can be in particular for meglumine salt of Compound B.

In some embodiments, the non-bile acid derived FXR agonist, e.g. Compound B as herein defined (e.g. in free form or a pharmaceutically acceptable salt thereof), is administered at a dose of about 50 mg twice daily, about 60 mg twice daily, about 80 mg twice daily, about 100 mg twice daily, about 140 mg twice daily, about 150 mg twice daily, about 180 mg twice daily, about 200 mg twice daily, about 220 mg twice daily, about 250 mg twice daily. Such regimens may be delivered orally. These doses can be in particular for meglumine salt of Compound B.

According to the invention, the CCR2/5 inhibitor, e.g. cenicriviroc (as hereinabove defined, e.g. cenicriviroc mesylate) is administered at a dose of about 50 mg, e.g. about 60 mg, e.g about 80 mg, e.g. about 100 mg, e.g. about 120 mg, e.g. about 140 mg, e.g. about 150 mg, e.g about 180 mg, e.g about 200 mg, e.g about 220 mg, e.g about 250 mg. Such doses may be for oral administration of CCR2/5 inhibitor, e.g. cenicriviroc (as hereinabove defined, e.g. cenicriviroc mesylate). Such doses may be for daily administration of CCR2/5 inhibitor, e.g. cenicriviroc (as hereinabove defined, e.g. cenicriviroc mesylate), twice daily administration or every two days administration, e.g. for daily oral administration.

In some aspects, the CCR2/5 inhibitor, e.g. cenicriviroc (as herein above defined, e.g cenicriviroc mesylate) is administered at a dose in a range of about 30 mg to about 250 mg, e.g about 50 mg to about 250 mg, e.g. about 100 mg to about 250 mg, e.g. about 10 mg to about 200 mg; e.g. about 100 mg to about 200 mg; e.g. about 30 mg to about 200 mg, e.g. about 50 mg to about 200 mg. Such doses may be for oral administration of CCR2/5 inhibitor, e.g. cenicriviroc (as hereinabove defined, e.g. cenicriviroc mesylate). Such doses may be for daily administration of CCR2/5 inhibitor, e.g. cenicriviroc (as hereinabove defined, e.g. cenicriviroc mesylate), twice daily administration or every two days administration, e.g. for daily oral administration.

In some embodiments, the CCR2/5 inhibitor, e.g. cenicriviroc (as hereinabove defined, e.g. cenicriviroc mesylate), is administered at a dose of about 50 mg delivered orally, about 60 mg delivered orally, about 80 mg delivered orally, about 100 mg delivered orally, about 120 mg delivered orally, about 140 mg delivered orally, about 150 mg delivered orally, about 180 mg delivered orally, about 200 mg delivered orally, about 220 mg delivered orally, about 250 mg delivered orally. Such doses may be particularly adapted for patients of weight between 50 and 120 kg, e.g. 70 and 100 kg.

In some embodiments, the CCR2/5 inhibitor, e.g. cenicriviroc (as hereinabove defined, e.g. cenicriviroc mesylate), is administered at a dose in a range of about 50 mg/day, e.g. about 60 mg/day, e.g about 80 mg/day, e.g. about 100 mg/day, e.g. about 120 mg/day, e.g. about 140 mg/day, e.g. about 150 mg/day, e.g. about 180 mg/day, e.g. about 200 mg/day, e.g. about 220 mg/day, e.g. about 250 mg/day. Such regimens may be delivered orally. Such regimens may be particularly adapted for patients of weight between 50 and 120 kg, e.g. 70 and 100 kg.

In some embodiments of the invention, the CCR2/5 inhibitor, e.g. cenicriviroc (as hereinabove defined, e.g. cenicriviroc mesylate), is administered at a dose of about 50 mg twice daily, about 60 mg twice daily, about 80 mg twice daily, about 100 mg twice daily, about 140 mg twice daily, about 150 mg twice daily, about 180 mg twice daily, about 200 mg twice daily, about 220 mg twice daily, about 250 mg twice daily. Such regimens may be delivered orally.

In one embodiment of the invention, the pharmaceutical combination, e.g fixed or free combination, comprises i) about 100 mg to about 250 mg of Compound B (as hereinabove defined, e.g. in free form or as a pharmaceutically acceptable salt thereof, e.g. meglumine salt) and ii) about 100 to about 250 mg of cenicriviroc (as hereinabove defined, e.g. cenicriviroc mesylate). For example, the pharmaceutical combination, e.g fixed or free combination, comprises i) about 100 mg of Compound B (as hereinabove defined, e.g. in free form or as a pharmaceutically acceptable salt thereof) and ii) about 150 mg of cenicriviroc (as hereinabove defined, e.g. cenicriviroc mesylate).

There is also provided pharmaceutical combinations containing, separate or together, (i) Compound A as herein defined (e.g. in free form or a pharmaceutically acceptable salt thereof); and (ii) a CCR2/5 inhibitor, e.g. cenicriviroc as herein defined (e.g. in free form or a pharmaceutically acceptable salt thereof), for simultaneous or sequential administration, wherein the ratio (μg/mg (microgram/milligram)) of Compound A to CCR2/5 inhibitor, e.g. cenicriviroc as hereinavove defined, is from about 3:100 to about 100:100; e.g. from about 10:100 to about 100:100; e.g. from about 20:100 to about 60:100; e.g. from about 10:100 to about 40:100; e.g. from about 5:100 to about 60:100; e.g. from about 5:100 to about 40:100. For example, the ratio (μg/mg (microgram/milligram)) of Compound A to CCR2/5 inhibitor, e.g. cenicriviroc as hereinavove defined, e.g. cenicriviroc mesylate, is about 3:100, about 5:100, about 10:100, e.g. about 40:100, e.g. about 60:100. These ratios are particularly adapted for pharmaceutical combinations comprising Compound A and cenicriviroc (as hereinavove defined, e.g. cenicriviroc mesylate)

In other embodiments, there are provided pharmaceutical combinations, containing, separate or together, (i) Compound B as herein defined (e.g. in free form or a pharmaceutically acceptable salt thereof, e.g. meglumine salt); and (ii) a CCR2/5 inhibitor, e.g. cenicriviroc (as hereinabove defined, e.g. cenicriviroc mesylate), for simultaneous or sequential administration, wherein the ratio (mg/mg) of Compound B to CCR2/5 inhibitor, e.g. cenicriviroc (as hereinabove defined), is about 0.5:1 to about 10:1, e.g. about 0.5:1 to about 8:1, e.g. about 0.5:1 to about 5:1; about 0.5:1 to about 3:1, e.g. about 1:1 to about 5:1, e.g. about 1:1 to about 3:1, e.g. about 1:1 to about 2:1, e.g. about 1:1. These ratios are particularly adapted for pharmaceutical combinations comprising Compound B (in free form or a pharmaceutically acceptable salt thereof, e.g. meglumine salt) and cenicriviroc (as hereinavove defined, e.g. cenicriviroc mesylate).

In specific embodiments of the inventions, the FXR agonist, e.g. non bile acid derived FXR agonist, e.g. Compound A or Compound B as herein defined (e.g. in free form or a pharmaceutically acceptable salt thereof, e.g. meglumine salt of Compound B) that is administered with an additional therapeutic agent, e.g. cenicriviroc (as hereinabove defined, e.g. cenicriviroc mesylate), is administered for a period of 3 months to lifelong, e.g. 6 months to lifelong, e.g. 1 year to lifelong, e.g. fora period of 3months to 1 year, e.g. 6 months to lifelong, e.g. for a period of 3 months, 6months or 1 year or for lifelong.

Kits for the Treatment of Fibrotic Disease or Disorder, e.g. a Liver Disease or Disorder

Accordingly, there are provided pharmaceutical kits comprising: a) a FXR agonist, e.g. non-bile acid derived FXR agonists, e.g. Compound A or Compound B (as hereinabove defined, e.g. in free form or as a pharmaceutically acceptable salt thereof; b) an additional therapeutic agent, e.g. a CCR2/5 inhibitor, e.g. cenicriviroc (as hereinabove defined, e.g. cenicriviroc mesylate);

and c) means for administering the FXR agonist (e.g. Compound A or B as herein defined) and the additional therapeutic agent (e.g. cenicriviroc (as hereinabove defined, e.g. cenicriviroc mesylate), to a subject affected by a liver disease or disorder; and optionally d) instructions for use.

In one embodiment of the invention, there is provided a combination package comprising a) at least one individual dose of a FXR agonist, e.g. non-bile acid derived FXR agonists, e.g. Compound A or Compound B as herein defined, e.g. in free form or as a pharmaceutically acceptable salt thereof; and b) at least one individual dose of an additional therapeutic agent as hereinabove defined, e.g. a CCR2/5 inhibitor, e.g. cenicriviroc (as hereinabove defined, e.g. cenicriviroc mesylate). The combination package may further comprise instructions for use.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

Example: In Vivo Efficacy Study of cenicriviroc in combination with Compound A (“NVP-CH-4”) and Compound B (“NVP-CH-5”) in the STAM Model of Non-alcoholic Steatohepatitis (Treatment period: 6 to 9 wks or 9 to12 wks)

The study involves 14-day-pregnant C57BL/6 mice. NASH was established by a single subcutaneous injection of 200pg streptozotocin (Sigma, USA) after birth and feeding with a high fat diet (HFD, 57% kcal fat, CLEA Japan, Japan) ad libitum after 4 weeks of age (day 28±2). Randomization of NASH mice into six groups of 12 mice at 6 weeks of age (day 42±2) and six groups of 12 mice at 9 weeks of age (day 63±2), the day before the start of treatment, respectively. NASH animals were dosed from age 6-9 weeks (Study 1), or from age 9-12 weeks (Study 2) with either: vehicle, cenicriviroc, Compound A (“NVP-CH-4”), Compound B (“NVP-CH-5”), cenicriviroc+Compound A or cenicriviroc+Compound B. A non-disease vehicle-control group of 12 mice was included in both Study 1 and Study 2. These animals were fed with a normal diet (CE-2; CLEA Japan) ad libitum.

PK samples were collected and stored at −60° C. Animals were dosed according to the dosing schedule below. Each animal was sacrificed 5 hours after last morning dose on the last day of study treatment (the evening dose of cenicriviroc or vehicle was administered that day due to sacrifice).

Dosing:
Cenicriviroc was prepared in 0.5% (w/v) methylcellulose with 1% Tween® 80 in sterile water for injection (USP).
Compound A and Compound B were prepared in 0.5% (w/v) methylcellulose (400 cPs) aqueous solution containing 0.5% (v/v) polysorbate 80, NF, in reverse osmosis water.
In general, cenicriviroc monotherapy was administered twice daily, 12 hours apart (AM and PM). For Compound A or Compound B monotherapy groups, drug was administered once daily in the morning with vehicle alone in the evening (12 hours after the morning dose).

Dosing Schedule:

| | | | | | |
|---|---|---|---|---|---|
| | | | | | AM/PM |
| | Dosing: | | AM | PM | Dose Volume |
| Group | Start-End | Treatment | Dose | Dose | (mL/kg) |

STUDY 1

| | | | | | |
|---|---|---|---|---|---|
| 1 | Week 6-9 | Non Disease- | AM Vehicle¹ | PM Vehicle² | 2.5 + 2.5 AM |
| | | vehicle | | | 5 PM |
| 2 | Week 6-9 | STAM-vehicle | AM Vehicle¹ | PM Vehicle² | 2.5 + 2.5 AM |
| | | | | | 5 PM |
| 3 | Week 6-9 | Cenicriviroc | 50 mg/kg | 50 mg/kg | 5/5 |
| 4 | Week 6-9 | Compound A | 0.1 mg/kg | PM Vehicle² | 5/5 |
| 5 | Week 6-9 | Compound B | 10 mg/kg | PM Vehicle² | 5/5 |
| 6 | Week 6-9 | Cenicriviroc + | 50 mg/kg + | 50 mg/kg | 2.5 + 2.5 AM |
| | | Compound A | 0.1 mg/kg | Cenicriviroc | 5 PM |
| 7 | Week 6-9 | Cenicriviroc + | 50 mg/kg + | 50 mg/kg | 2.5 + 2.5 AM |
| | | Compound B | 10 mg/kg | Cenicriviroc | 5 PM |

STUDY 2

| | | | | | |
|---|---|---|---|---|---|
| 8 | Week 9-12 | Non Disease- | AM Vehicle¹ | PM Vehicle² | 2.5 + 2.5 AM |
| | | vehicle | | | 5 PM |
| 9 | Week 9-12 | STAM-vehicle | AM Vehicle¹ | PM Vehicle² | 2.5 + 2.5 AM |
| | | | | | 5 PM |
| 10 | Week 9-12 | Cenicriviroc | 50 mg/kg | 50 mg/kg | 5/5 |
| 11 | Week 9-12 | Compound A | 0.1 mg/kg | PM Vehicle² | 5/5 |
| 12 | Week 9-12 | Compound B | 10 mg/kg | PM Vehicle² | 5/5 |
| 13 | Week 9-12 | Cenicriviroc + | 50 mg/kg + | 50 mg/kg | 2.5 + 2.5 AM |
| | | Compound A | 0.1 mg/kg | Cenicriviroc | 5 PM |
| 14 | Week 9-12 | Cenicriviroc + | 50 mg/kg + | 50 mg/kg | 2.5 + 2.5 AM |
| | | Compound B | 10 mg/kg | Cenicriviroc | 5 PM |

¹AM vehicle = low pH vehicle followed by neutral pH vehicle (2.5 ml/kg each for total volume 5 ml/kg);
²PM vehicle = low pH vehicle only (5 ml/kg volume).

Measurements:
The following parameters were measured or monitored daily: individual body weight, survival, clinical signs and behavior of mice.
Pharmacokinetic measurements: PK samples were collected from 4 animals per time point per compound. PK samples were for Compound A and Compound B at hours 1 and 24 on Day 6 (n=4 per timepoint) for both monotherapy and combination groups. PK samples were taken for Cenicriviroc on Day 10 at hours 2 and 12 (n=4 per timepoint) for both monotherapy and combination groups. Only one PK sample was collected per animal using the first 8 animals per group, per the schedule below:

| DAY 6 | | | | Cenicriviroc + | Cenicriviroc + |
|---|---|---|---|---|---|
| (Study 1 and 2) | Cenicriviroc | Compound A | Compound B | Compound A | Compound B |
| 1 hr | 0 | n = 4; 50 μL | n = 4; 50 μL | n = 4; 50 μL | n = 4; 50 μL |
| | | whole blood | whole blood | wholeblood | whole blood |
| | | | | Compound A only | Compound B only |
| 24 hr | 0 | n = 4; 50 μL | n = 4; 50 μL | n = 4; 50 μL | n = 4; 50 μL |
| | | whole blood | whole blood | whole blood | whole blood |
| | | | | Compound A only | Compound B only |

| | | | | Cenicriviroc + | Cenicriviroc + |
|---|---|---|---|---|---|
| DAY 10 | Cenicriviroc | Compound A | Compound B | Compound A | Compound B |
| (Study 1 and 2) | (gr. 3 and 10) | (gr. 4 and 11) | (gr. 5 and 12) | (gr. 6 and 13) | (gr. 7 and 14) |
| 2 hr | n = 4; 50 μL | 0 | 0 | n = 4; 50 μL | n = 4; 50 μL |
| | whole blood | | | whole blood | whole blood |
| | | | | Cenicriviroc | Cenicriviroc |
| 12 hr | n = 4; 50 μL | 0 | 0 | n = 4; 50 μL | n = 4; 50 μL |
| | whole blood | | | whole blood | whole blood |
| | | | | Cenicriviroc | Cenicriviroc |

End of Treatment Measurements: Mice were sacrificed at 9 weeks of age (study 1) or at 12 weeks of age (study 2). The 8 NASH animals that that did not receive any treatment or vehicle were sacrificed at week 9 as a ‘baseline’ in order for comparisons of any fibrosis regression events observed in treated animals.

The following samples were collected: plasma, liver (fresh liver samples for gene expression analysis were collected at 5 hr post the last morning (AM) dose for each animal), stool. Organ weight was measured.

The following biochemical assays were performed: Non-fasting blood glucose in whole blood by Life Check (Eidia, Japan); serum ALT by FUJI DRI-CHEM (Fujifilm, Japan); serum triglyceride; serum MCP-1, RANTES (CCLS) and MIP-1α/MIP-1 quantification by a commercial ELISA kit; liver triglyceride by Triglyceride E-test kit (Wako, Japan); liver hydroxyproline quantification by hydrolysis method; histological analyses for liver section; HE staining and estimation of NAFLD Activity score; Sirius-red staining and estimation of fibrosis area (with and without perivascular space subtracted); oil red staining and estimation of fat deposition area; F4/80 immunohistochemistry staining and estimation of inflammation area; alpha-SMA immunohistochemistry staining and estimation of α-SMA positive area Gene expression assays using total RNA from the liver.

Real-time RT-PCR analyses were performed for: MCP-1, MIP-1α/β, RANTES, Emr1, CD68, TGF-β1, CCR2/5, TIMP-1, Cola1A1, TNF, IL-10, MMP-9, α-SMA and CX3CR1/CX3CL1, SHP (small heterodimer partner), BSEP (bile salt export pump), Cyp8b1.

Statistical tests were performed using one-way ANOVA followed by Dunnett's test and the Mann-Whitney test, as appropriate, for the multiple group comparisons. P values <0.05 are considered statistically significant.

Results:

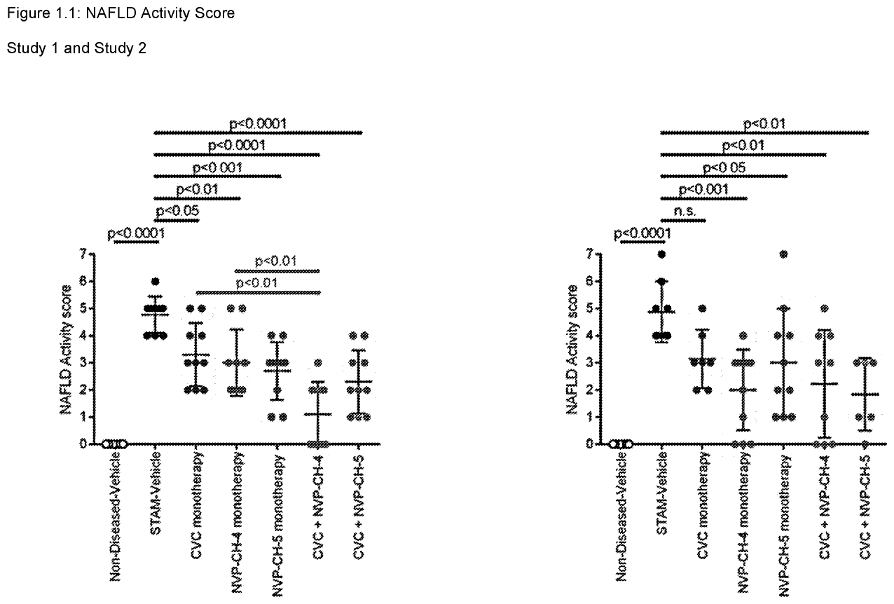

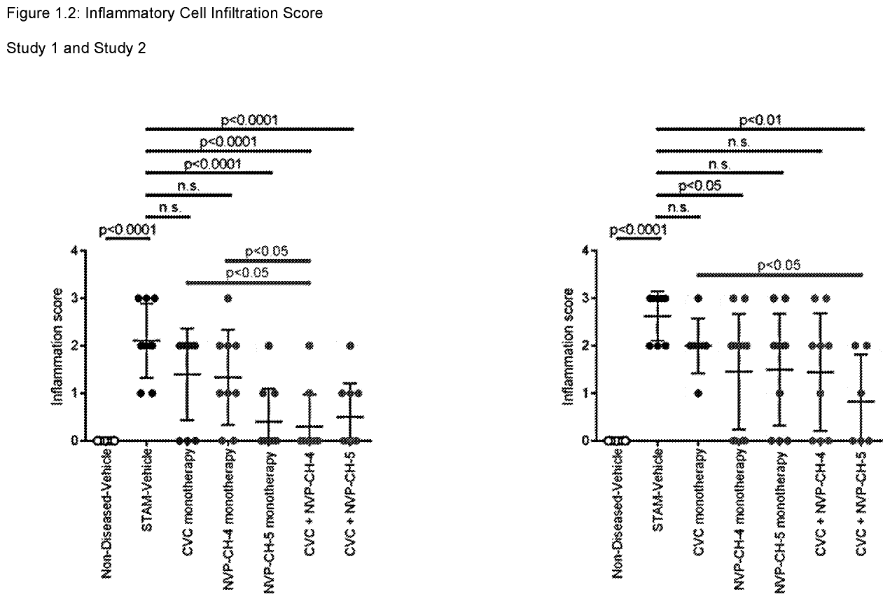

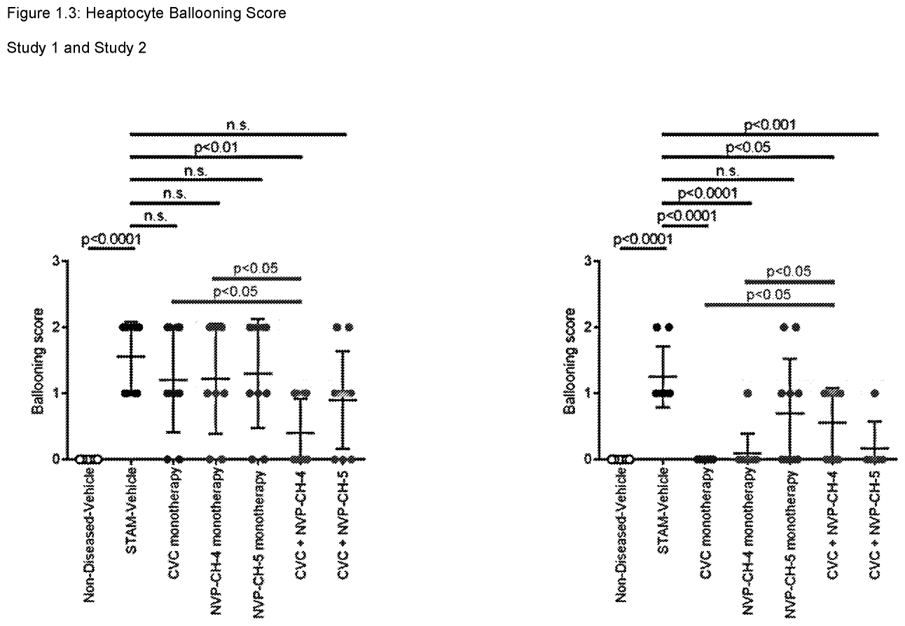

As described in FIG. 1.1:

Study 1: The CVC monotherapy, Compound A monotherapy, Compound B monotherapy, the CVC+Compound A and the CVC+Compound B groups showed significant reductions in NAS compared with the STAM-Vehicle group. The CVC+Compound A group showed a significant reduction in NAS compared with both the CVC monotherapy and the Compound A monotherapy groups demonstrating a synergistic effect of the treatment combination.

Study 2: The Compound A monotherapy, the Compound B monotherapy, the CVC+Compound A and the CVC+Compound B groups showed significant reductions in NAS compared with the STAM-Vehicle group.

As described in FIG. 1.2:

Study 1: The Compound B monotherapy, the CVC+Compound A and the CVC+Compound B groups showed significant reductions in the inflammatory cell infiltration score compared with the STAM-Vehicle group. The CVC+Compound A group showed a significant reduction in NAS compared with both the CVC monotherapy and the Compound A monotherapy groups demonstrating a synergistic effect of the treatment combination.

Study 2: All monotherapy treatments and the CVC+Compound B group showed a clear trend in the reduction of the inflammatory cell infiltrate score. The CVC+Compound B group showed a statistically singificant reduction demonstrating a synergistic effect of the treatment combination.

As described in FIG. 1.3:

Study 1: All monotherapy groups showed a trend in the reduction of the ballooning score. The CVC+Compound A group showed a significant reduction in NAS compared with both the CVC monotherapy and the Compound A monotherapy groups demonstrating a synergistic effect of the treatment combination.

Study 2: All monotherapy groups with the exception of Compound B and all combination groups showed a significant reduction of the ballooning score.

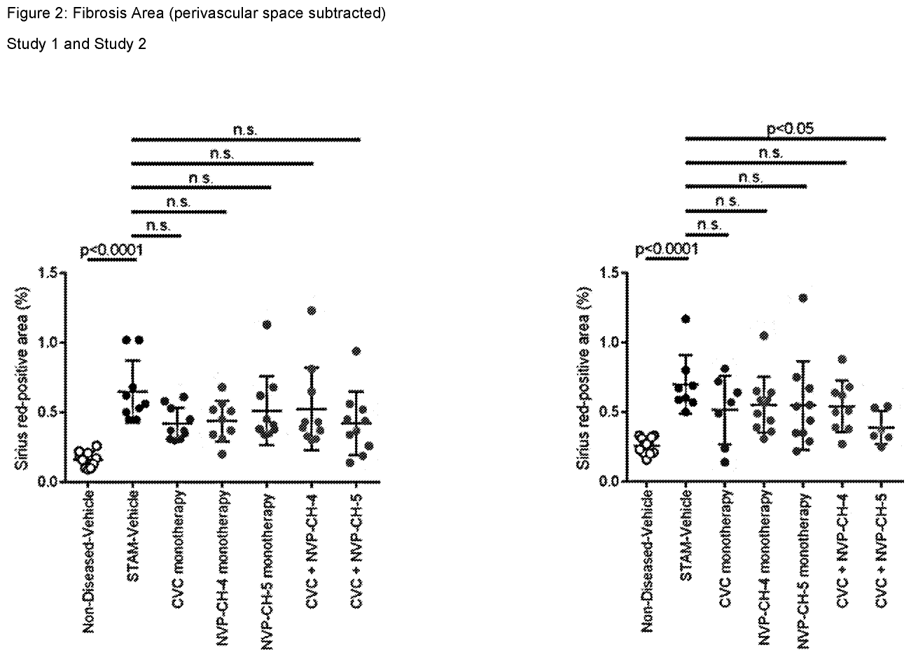

As described in FIG. 2:

Study 1: All monotherapy groups and all combination groups showed a trend in the reduction of the fibrosis area.

Study 2: All monotherapy groups and all combination groups showed a trend in the reduction of the fibrosis area. The CVC+Compound B group showed a significant decrease in fibrosis area compared with the STAM-Vehicle group and a clear trend to further reduce the fibrosis area compared to the CVC monotherapy and Compound B monotherapy demonstrating a synergistic effect of the combination.

1.-13. (canceled)

14. A pharmaceutical combination containing a non-bile acid derived FXR agonist and one or more additional therapeutic agent, for simultaneous, sequential or separate administration, wherein the additional therapeutic agent is a CCR2/5 inhibitor, e.g. cenicriviroc, and wherein the FXR agonist is 2-[3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid, a stereoisomer, an enantiomer, a pharmaceutically acceptable salt, prodrug, and/or ester thereof or an amino acid conjugate thereof.

15. A combination according to claim 14 for use in treating or preventing a fibrotic or cirrhotic disease or disorder, e.g. a liver disease or disorder, e.g. a chronic liver disease or disorder.

16. Combination according to claim 14, wherein the FXR agonist, is to be administered at a dose in a range of about 3 μg to about 100 μg.

17. Combination according to claim 14, wherein cenicriviroc in free form or as a pharmaceutically acceptable salt, solvate, prodrug or ester thereof, is to be administered at a dose in a range of about 50 mg to about 250 mg.

18. Combination according to claim 14 which is a fixed dose combination.

19. Combination according to claim 14 which is a free combination.

20. Use of a combination according to claim 14, in the manufacture of a medicament for treating or preventing a fibrotic, cirrhotic disease or disorder, e.g. a liver disease or disorder, e.g. a chronic liver disease, e.g. a liver disease or disorder selected from the group consisting of cholestasis, intrahepatic cholestasis, estrogen-induced cholestasis, drug-induced cholestasis, cholestasis of, pregnancy, parenteral nutrition-associated cholestasis, primary biliary cirrhosis (PBC), primary sclerosing cholangitis (PSC), progressive familiar cholestasis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, dyslipidemia, atherosclerosis, diabetes, diabetic nephropathy, colitis, newborn jaundice, prevention of kernicterus, veno-occlusive disease, portal hypertension, metabolic syndrome, hypercholesterolemia, intestinal bacterial overgrowth, erectile dysfunction, progressive fibrosis of the liver caused by any of the diseases above or by infectious hepatitis; e.g. NAFLD, NASH, liver fibrosis, or PBC.

21. A method for preventing, delaying or treating a liver disease or disorder as defined in claim 20, in a patient in need therefor, comprising administering a therapeutically effective amount of combination as defined in claim 14.
